(19) United States
(12) Patent Application Publication
(10) Pub. No.: US 2006/0177486 A1
(43) Pub. Date: Aug. 10, 2006

(54) Enzymatically synthesized marine phospholipids

(75) Inventors: Inge Bruheim, Volda (NO); Hogne Hallaraker, Volda (NO); Mikko Griinari, Espoo (FI); Erik Fuglseth, Molde (NO); Per Christian Saebo, Volda (NO)

Correspondence Address: Medlen & Carroll, LLP;Suite 350, 101 Howard Street, San Francisco, CA, 94105, US

(73) Assignee: Natural ASA, Lysaker (NO)

(21) Appl. No.: 11/282,033

(22) Filed: Nov. 17, 2005

Related U.S. Application Data
(60) Provisional application No. 60/628,833, filed on Nov. 17, 2004.
(60) Provisional application No. 60/706,525, filed on Aug. 9, 2005.
(60) Provisional application No. 60/717,871, filed on Sep. 15, 2005.

(51) Int. Cl. A23K 1/165; C12P 13/00; C12P 9/00

(52) U.S. Cl. 424/442; 435/128; 435/131

(57) ABSTRACT

This invention discloses an improved enzymatic process, under organic solvent free conditions, for the incorporation of fatty acids such as omega-3 fatty acids into phospholipids. The rate of transesterification is increased 4 times by adding a base to the reaction mixture, typically an amine. The invention also discloses novel phospholipid compositions as well as novel use of the phospholipid compositions as a food supplement, a fish feed, animal feed and human food. In addition to methods for enriching prey organisms used in aquaculture, methods of reducing arachidonic acid levels in mammalian plasma/red blood cells and methods for increasing DHA levels in the mammalian brain.

This application claims the benefit of U.S. Provisional Patent Application Ser. No. 60/628,833, filed Nov. 17, 2004; U.S. Provisional Patent Application Ser. No. 60/706,525, filed Aug. 9, 2005; and U.S. Provisional Patent Application Ser. No. 60/717,871 filed Sep. 15, 2005, each are incorporated herein by reference.

### FIELD OF THE INVENTION

The present invention relates to processes for making structured phospholipids containing desired fatty acid residues, especially DHA and EPA, compositions resulting from the processes, and their use.

### BACKGROUND OF THE INVENTION

A phospholipid consists of glycerol esterified with two fatty acyl groups and one phosphate or esterified phosphate group. For some applications it is desirable to exchange the acyl groups in the phospholipid in order to improve emulsification properties, physiological value and nutritional value of the phospholipid.

Many recent reports indicate that the fatty acyl moiety on the sn-1 position in phospholipids can be replaced using different types of hydrolases, such as specific and non-specific lipases with broad substrate specificity (P. Adlercreutz, A-M Lyberg and D. Adlercreutz, Eur. J. Lipid. Sci. Technol. 105 (2003) 638-645; WO9103564; S. Doig and R. M. M. Diks; Eur. J. Lipid Sci. Technol. 105 (2003) 359; U.S. Pat. No. 6,537,787). Transesterification in the presence of an organic solvent resulted in phospholipids having eicosapentaenoic acid (EPA, 20:5) and decosahexaenoic acid (DHA, 22:6) content of around 50% of the total fatty acid (U.S. Pat. No. 6,537,787). However, the level of byproducts formed such as lyso-phospholipids were not disclosed. Haraldsson et. al. published a method for the enzymatic transesterification of pure phosphatidylcholine (PC) obtained from egg. Although, the synthesis was performed under organic solvent free conditions, chloroform was used to isolate the final product (G. G. Haraldsson, A. Thorarensen, JAOCS 75 (1999) 1143-1149). The process required 72 hours to incorporate 58% EPA into PC. However, extensive side reactions resulted in only 39% PC (w,w), 44% lysophosphatidylcholine (LPC) and 17% glycerophosphatidylcholine (GPC). It was also disclosed a phospholipid composition containing 16% DHA with no indications of the level of LPC and GPC present. All of the previous published or patented methods suffer from one or more of the following drawbacks: requiring the presence of a non-food compatible solvent; requiring the use of purified staring materials; yielding unwanted phospholipid side products; or having not been demonstrated above gram scale.

Accordingly, what is needed is an enzymatic method capable of incorporating fatty acids or esters, preferably polyunsaturated fatty acids, most preferably DHA and EPA into a low-cost lecithin starting material under organic solvent free conditions with a high yield. The marine phospholipids should be less expensive and have the same or improved quality as compared to a naturally occurring marine phospholipids.

### SUMMARY OF THE INVENTION

In a first aspect, the invention provides an improved method for the enzymatic transesterification of phospholipids by adding an effective amount of a base to the reaction mixture. It is contemplated that the addition of the base enhances the rate of transesterification and reduces the inhibition of the immobilized enzyme. In a further aspect of the invention, the invention provides a phospholipid product characterized by having 20-100% DHA in position 1 (10-50% DHA in phospholipid molecule). In a further aspect of the invention, a safe and palatable marine phospholipid is obtained. In yet a further aspect, the invention provides the use of the above composition for enriching prey organisms used in aquaculture for feeding fish at the larvae and post-larvae stage. In yet another aspect, the invention provides the use of the above composition for providing bioavailable DHA to mammals. In yet another aspect, the invention provides the use of the above composition for reducing plasma levels of arachidonic acids (AA) and thereby having the potential to reduce inflammation. In addition, the compositions find use for supplementing infant formula, animal feed and food products for humans. In addition, the above compositions find use as pharmaceutical compositions and as a food supplements.

Accordingly, in some embodiments, the present invention provides a process for modifying phospholipid material which comprises exchanging acyl groups in a phospholipid by enzymatic exchange with a free fatty acid or ester, the reaction mixture comprising an immobilized lipase and a cationic compound, wherein the cationic compound enhances the enzymatic activity of the immobilized lipase. In some embodiments, the cationic compound is an organic molecule with an amine functional group. In some preferred embodiments, the cationic compound is present in the range of 0.1-10% relative to the phospholipid (w/w). In some embodiments, the organic molecule containing an amine functional group is triethylamine or ethanolamine. In further embodiments, the acyl donor is a fatty acid ethyl ester containing EPA or DHA. In still further embodiments, the phospholipid starting material is a naturally occurring soybean lecithin. In some embodiments, the reaction is substantially solvent-free.

In some embodiments, the foregoing methods further comprise the step of supplementing a food product with the modified phospholipid. In some embodiments, the methods further comprise the step of formulating a pharmaceutical composition with the modified phospholipid. In some embodiments, the methods further comprise the step of supplementing an animal feed with the modified phospholipid. In some embodiments, the methods further comprise the step of supplementing an infant formula with the modified phospholipid. In some embodiments, the methods further comprise the step of formulating the modified phospholipids for oral administration.

In some embodiments, the present invention provides a composition produced by the foregoing methods, wherein the composition comprises phospholipids having a DHA or EPA residue at position 1 of the phospholipid. In some embodiments, the present invention provides an oral delivery vehicle comprising the composition. In some embodiments, the present invention provides a food product comprising the composition. In some embodiments, the present invention provides a pharmaceutical composition comprising the composition. In some embodiments, the present invention provides an animal feed comprising the composition.

In some embodiments, the present invention provides compositions comprising phospholipids having the following structure: 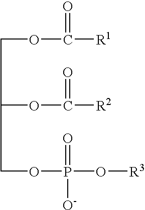
wherein R1 is a fatty acid, R2 is OH or a fatty acid, and R3 is H or choline, ethanolamine, inositol or serine, said composition having at least 5 to 10% of a combination of DHA and EPA at position R1 and being substantially free of EPA and DHA at position R2. In some embodiments, the composition contains from about 10% DHA to about 50% DHA at position R1. In some embodiments, the composition contains from about 5 to 10% DHA to about 40% DHA at position R1. In some embodiments, the composition contains from about 10% DHA to about 30% DHA at position R1. In some embodiments, the composition of Claim 18, wherein said composition contains from about 10% DHA to about 20% DHA at position R1. In some embodiments, the composition contains from about 10% EPA to about 50% EPA at position R1. In some embodiments, the composition of Claim 18, wherein said composition contains from about 5 to 10% EPA to about 40% EPA at position R1. In some embodiments, the composition contains from about 10% EPA to about 30% EPA at position R1. In some embodiments, the composition contains from about 10% EPA to about 20% EPA at position R1. In some embodiments, the composition contains from about 15% DHA and/or EPA to about 50% DHA and/or EPA at position R1. In some embodiments, the composition contains from about 15% DHA and/or EPA to about 40% DHA and/or EPA at position R1. In some embodiments, the composition contains from about 15% DHA and/or EPA to about 30% DHA and/or EPA at position R1. In some embodiments, the composition contains from about 15% DHA and/or EPA to about 20% DHA and/or EPA at position R1. In some embodiments, the composition contains from about 20% DHA and/or EPA to about 50% DHA and/or EPA at position R1. In some embodiments, the composition contains from about 20% DHA and/or EPA to about 40% DHA and/or EPA at position R1. In some embodiments, the composition contains from about 20% DHA and/or EPA to about 30% DHA and/or EPA at position R1. In some embodiments, the composition contains from about 15% DHA and/or EPA to about 25% DHA and/or EPA at position R1.

In some embodiments, the composition is at least about 50% acylated at positions R1 and R2. In some embodiments, the composition contains from about 5% to about 75% of a linoleic acid isomer residue at position R2. In some embodiments, the composition contains from about 5% to about 50% of a linoleic acid isomer residue at position R2. In some embodiments, the linoleic acid isomer residue is selected from the group consisting of 9,12-ocadecadienoic acid, 9,11-ocadecadienoic acid, 10,12-ocadecadienoic acid, 8,10-octadecadienoic acid, and 11,13-octadecodienoic acid and combinations thereof. In some embodiments, the composition comprises less than about 5% EPA or DHA a position R2. In some embodiments, the composition comprises less than about 1% EPA or DHA a position R2. In some embodiments, the foregoing compositions provide increased bioavailability.

In some embodiments, the composition is substantially free of organic solvents. In some embodiments, a food product is provided that is safe to be taken orally by humans in a concentrated form comprising the foregoing compositions. In some embodiments, an animal feed is provided comprising the foregoing compositions. In some embodiments, a pharmaceutical composition is provided comprising the composition of Claim 18.

In some embodiments, the present invention provides compositions comprising synthetic phospholipids having the following structure:

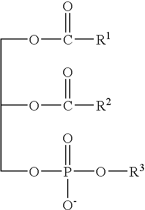

wherein R1 is a fatty acid, R2 is OH or a fatty acid, and R3 is H or choline, ethanolamine, inositol or serine, said composition characterized in having high palatability in terms of at least one of smell, taste, aftertaste, and mouthfeel or combinations thereof. In some embodiments, the high palatability is in comparison to at least one of naturally extracted marine phospholipids and synthetic phospholipids prepared with organic solvents. In some embodiments, the palatability is determined by a panel of human subjects.

In some embodiments, the present invention provides a safe and palatable synthetic marine phospholipid composition characterized in being substantially free of at least one of organic solvents and volatile organic compounds.

In some embodiments, the present invention provides compositions providing increased bioavailability of long chain fatty acids comprising phospholipids having the following structure: 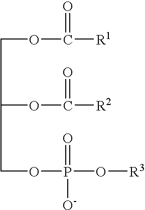
wherein R1 is a fatty acid, R2 is OH or a fatty acid, and R3 is H or choline, ethanolamine, inositol or serine, said composition enriched for DHA or EPA at position R1 as compared to position R2. In some embodiments, the composition has at least 10% DHA at position R1 and being substantially free of EPA and DHA at position R2.

In some embodiments, the present invention provides methods of increasing the bioavailability of EPA or DHA comprising:
providing phospholipids having the following structure: 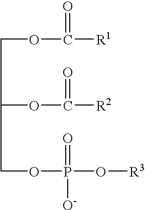
   wherein R1 is a fatty acid, R2 is OH or a fatty acid, and R3 is H or choline, ethanolamine, inositol or serine, said composition enriched for DHA or EPA at position R1 as compared to position R2 and administering said composition to a subject under conditions such that bioavailabilty of EPA or DHA to said subject is increased as compared to compositions enriched for EPA or DHA at position R2. In some embodiments, the present invention contemplates using the compositions described in more detail above in this method.

In some embodiments, the present invention provides methods of treating inflammation in a subject comprising: a) providing a phospholipid composition comprising DHA, EPA or a combination thereof, and b) administering said phospholipids composition to a subject under conditions such that inflammation in said subject is reduced. In some embodiments, the phospholipid composition is one of the compositions described in detail above. In some embodiments, the phospholipid composition is extracted from natural sources. In some embodiments, the subject is a human. In some embodiments, the subject is an animal. In some embodiments, the present invention provides methods of producing prey organisms for use in aquaculture, said method comprising cultivating said organisms during at least part of their life cycle in an aqueous medium comprising the compositions described in detail above. In some embodiments, the prey organisms are rotifers. In some embodiments, the prey organisms are artemia.

### BRIEF DESCRIPTION OF THE FIGURES

FIG. 1 is a graph showing the growth rate of gilthead seabream fed prey organisms enriched with 6 different diets (control and NAT501-NAT505).

FIG. 2 is a table providing a summary of quality parameters determined in both small and big grades of fish at 2 g. 200 fish from each group (small and big grades) were taken for external examination of which 96 fish were taken for X-rays.

### DEFINITIONS

As used herein, “phospholipid” refers to an organic compound having the following general structure: 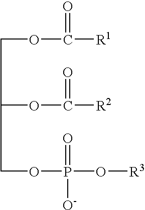
wherein R1 is a fatty acid residue, R2 is a fatty acid residue or —OH, and R3 is a —H or nitrogen containing compound choline (HOCH₂CH₂N⁺(CH₃)₃OH⁻), ethanolamine (HOCH₂CH₂NH₂), inositol or serine. R1 and R2 cannot simultanously be OH. When R3 is an H, the compound is a diacylglycerophosphate, while when R3 is a nitrogen-containing compound, the compound is a phosphatide such as lecithin, cephalin, phosphatidyl serine or plasmalogen. The R1 site is herein referred to as position 1 of the phospholipid, the R2 site is herein referred to as position 2 of the phospholipid, and the R3 site is herein referred to as position 3 of the phospholipid.

As used herein, the term omega-3 fatty acid refers to polyunsaturated fatty acids that have the final double bond in the hydrocarbon chain between the third and fourth carbon atoms from the methyl end of the molecule. Non-limiting examples of omega-3 fatty acids include, but are not limited to 5,8,11,14,17-eicosapentaenoic acid (EPA), 4,7,10,13,16,19-docosahexanoic acid (DHA) and 7,10,13,16,19-docosapentanoic acid (DPA).

As used herein, the term “physiologically acceptable carrier” refers to any carrier or excipient commonly used with pharmaceuticals. Such carriers or excipients include, but are not limited to, oils, starch, sucrose and lactose.

As used herein, the term “oral delivery vehicle” refers to any means of delivering a pharmaceutical orally, including, but not limited to, capsules, pills, tablets and syrups.

As used herein, the term “food product” refers to any food or feed suitable for consumption by humans, non-ruminant animals, or ruminant animals. The “food product” may be a prepared and packaged food (e.g., mayonnaise, salad dressing, bread, or cheese food) or an animal feed (e.g., extruded and pelleted animal feed or coarse mixed feed). “Prepared food product” means any pre-packaged food approved for human consumption.

As used herein, the term “foodstuff” refers to any substance fit for human or animal consumption.

As used herein, the term “functional food” refers to a food product to which a biologically active supplement has been added.

As used herein, the term “infant food” refers to a food product formulated for an infant such as formula.

As used herein, the term “elderly food” refers to a food product formulated for persons of advanced age.

As used herein, the term “pregnancy food” refers to a food product formulated for pregnant women.

As used herein, the term “nutritional supplement” refers to a food product formulated as a dietary or nutritional supplement to be used as part of a diet.

As used herein, the term “medium chain fatty acyl residue” refers to fatty acyl residues derived from fatty acids with a carbon chain length of equal to or less than 14 carbons.

As used herein, the term “long chain fatty acyl residue” refers to fatty acyl residues derived from fatty acids with a carbon chain length of greater than 14 carbons.

As used herein, the term “cationic compound” refers to compounds that are positively charged or form positively charged compounds in contact with other molecules (e.g. water). As used herein, the term “base” refers to compounds that have the ability to pick up protons and/or to donate pair of electrons.

By “safe for oral administration” it is meant that the compositions are substantially free of organic solvents and undesirable volatile organic compounds.

As used herein, the term “extracted marine phospholipid” refers to a composition characterized by being obtained from a natural source such as krill or fish meal.

### DESCRIPTION OF THE INVENTION

The present invention disclosed relates to an improved method for the transesterification of phospholipids with a free fatty acid or an ester under substantially solvent free conditions. The reaction is catalyzed by an immobilized lipase, such as Thermomyces Lanuginosus (TL-IM) in the presence of a small organic molecule, preferably a basic compound. In some preferred embodiments, the basic compound is a cationic compound which contains an amine functional group. In further preferred embodiments, the basic compound can be, e.g., triethylamine, ethanolamine, sodium methoxide or caffeine. In preferred embodiments, the cationic compound is included in the reaction mixture in the range of 0.1-10%, preferably in the range of 1-5%, (w/w) relative to the amount of phospholipid.

This invention discloses that by adding 3% (w/w) triethylamine or 3% (w/w) ethanolamine to a mixture consisting of TL-IM from Novozymes (Bagsvaerd, Denmark), fatty acid ethyl esters and phospholipids the rate of transesterification increased more than 4 or 2 times, respectively. Furthermore, addition of an amine allows for a lower lipase dosage (33% reduction), obtaining the same level of transesterification in the same amount of time. Furthermore, phospholipids may inhibit and reduce the activity of the enzymes as reported by others (Y. Watanabe, Y. Shimada, A Sugihara and Y. Tominage, J. Mol. Cat. B: Enzymatic 17 (2002) 151-155). It was found that without amine addition, lipases such as TL-IM, RM-IM and Novozyme 435 could only be used once. However, by adding 3% ethanolamine to the reaction mixture the deactivation of the enzyme slowed down, thereby allowing the enzyme to be reused for more than one batch. In this way the amine helps lowering the cost of production.

The present invention is not limited to any particular mechanism of action. Indeed, an understanding of the mechanism of action is not necessary to practice the present invention. Nevertheless, it is contemplated that the purpose of adding an amine to the reaction mixture is to prevent the phospholipids from interacting with the active sites on the enzyme carrier. Active sites may be left on the carrier after the immobilization procedure due to the large size and sterically demanding nature of the enzyme molecule. It is also a benefit that the additive has a rapid rate of diffusion in order to be able to compete efficiently with the phosphatides or other compounds present for these active sites. In the case were the enzymes are immobilized on silica, free silanol will be the predominant active group and amines are therefore particular suitable. However, enzymes may be immobilized on other carriers such as polymers or ion exchange resins and in that case other compounds may be more suitable depending on the chemical properties of the unreacted surface.

Accordingly, in preferred embodiments, the present invention utilizes a phospholipid, preferably a phosphatide such as lecithin, in an enzymatic reaction so that the fatty acid in position 1 of the phospholipid is replaced with a desired fatty acid residue. The present invention is not limited to the use of any particular phospholipid. Indeed, the use of a variety of phospholipids is contemplated. In some embodiments, the phospholipid is a phosphatidic or lysophosphatidic acid. In more preferred embodiments, the phospholipid is a mixture of phosphatides such as phosphatidylcholine, phospatidylethnolamine, phosphatidylserine and phosphatidylinositol. The present invention is not limited to the use of any particular source of phospholipids. In some embodiments, the phospholipids are from soybeans, while in other embodiments, the phospholipids are from eggs. In particularly preferred embodiments, the phospholipids utilized are commercially available, such as Alcolec 40P® from American Lecithin Company Inc (Oxford, Conn., USA). However, this invention discloses that the rate of transesterification is dependent on the purity of the phospholipid starting material i.e. the more pure the PC fraction the faster the reaction. The reduced reactivity for 40% PC versus 99% PC can to some extent be compensated by adding a base such as triethylamine to the reaction mixture.

In preferred embodiments, the replacement (e.g., by transesterification) of the phospholipid fatty acids with a desired fatty acid or the addition (e.g. esterification) is catalyzed by a lipase. The present invention is not limited to the use of any particular lipase. Indeed, the use of a variety of lipases is contemplated, including, but not limited to, the aforementioned Thermomyces Lanuginosus lipase, Rhizomucor miehei lipase, Candida Antarctica lipase, Pseudomonas fluorescence lipase, and Mucor javanicus lipase. It is contemplated that a variety of desired fatty acids may be substituted onto the phospholipids utilized in the process of the present invention, especially fatty acids that are not initially present in the starting phospholipid composition. Indeed, the incorporation of a variety of long chain and medium chain fatty acid residues is contemplated, including, but not limited to decanoic acid (10:0), undecanoic acid (11:0), 10-undecenoic acid (11:1), lauric acid (12:0), cis-5-dodecanoic acid (12:1), tridecanoic acid (13:0), myristic acid (14:0), myristoleic acid (cis-9-tetradecenoic acid, 14:1), pentadecanoic acid (15:0), palmitic acid (16:0), palmitoleic acid (cis-9-hexadecenoic acid, 16:1), heptadecenoic acid (17:1), stearic acid (18:0), elaidic acid (trans-9-octadecenoic acid, 18:1), oleic acid (cis-9-octadecenoic acid, 18:1), nonadecanoic acid (19:0), eicosanoic acid (20:0), cis-11-eicosenoic acid (20:1), 11,14-eicosadienoic acid (20:2), heneicosanoic acid (21:0), docosanoic acid (22:0), erucic acid (cis-13-docosenoic acid, 22:1), tricosanoic acid (23:0), tetracosanoic acid (24:0), nervonic acid (24:1), pentacosanoic acid (25:0), hexacosanoic acid (26:0), heptacosanoic acid (27:0), octacosanoic acid (28:0), nonacosanoic acid (29:0), triacosanoic acid (30:0), vaccenic acid (t-11-octadecenoic acid, 18:1), tariric acid (octadec-6-ynoic acid, 18:1), and ricinoleic acid (12-hydroxyoctadec-cis-9-enoic acid, 18:1) and ω3, ω6, and ω9 fatty acyl residues such as 9,12,15-octadecatrienoic acid (α-linolenic acid) [18:3, ω3]; 6,9,12,15-octadecatetraenoic acid (stearidonic acid) [18:4, ω3]; 11,14,17-eicosatrienoic acid (dihomo-α-linolenic acid) [20:3, ω3]; 8,11,14,17-eicosatetraenoic acid [20:4, ω3], 5,8,11,14,17-eicosapentaenoic acid [20:5, ω3]; 7,10,13,16,19-docosapentaenoic acid [22:5, ω3]; 4,7,10,13,16,19-docosahexaenoic acid [22:6, ω3]; 9,12-octadecadienoic acid (linoleic acid) [18:2, ω6]; 6,9,12-octadecatrienoic acid (γ-linolenic acid) [18:3, ω6]; 8,11,14-eicosatrienoic acid (dihomo-γ-linolenic acid) [20:3 ω6]; 5,8,11,14-eicosatetraenoic acid (arachidonic acid) [20:4, ω6]; 7,10,13,16-docosatetraenoic acid [22:4, ω6]; 4,7,10,13,16-docosapentaenoic acid [22:5, ω6]; 6,9-octadecadienoic acid [18:2, ω9]; 8,11-eicosadienoic acid [20:2, ω9]; and 5,8,11-eicosatrienoic acid (Mead acid) [20:3, ω9]. Moreover, acyl residues may be conjugated, hydroxylated, epoxidated or hydroxyepoxidated acyl residues. In preferred embodiments, the desired fatty acids are provided as free fatty acids or esters. In some particularly preferred embodiments, the fatty acids are omega-3 fatty acids such as DHA or EPA. These fatty acids may be derived from a variety of sources, including, but not limited to fish oil obtained from species such as: tuna, herring, mackerel and sardines caught in cold waters. Also, preferred sources of EPA/DHA are oils extracted from microbial cells such as algae and cod liver oil.

The process of the present invention provides compositions comprising phospholipids with a desired fatty acid at position 1. Accordingly, the composition comprises phospholipids with the following structure: 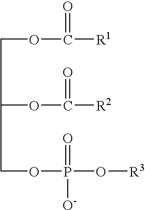
wherein R1 is one of the fatty acid residues described above, preferably DHA or EPA, R2 is OH or a fatty acid present in the initial phospholipid composition, and R3 is H or a nitrogen containing compound such as choline, serine or ethanolamine; or one without such as inositol. In some preferred embodiments, the phospholipid compositions of the present invention comprise a mixture of phospholipids with different fatty acids at position 1. Accordingly, in some embodiments, the overall fatty acid composition is from about 5-90% of one or more desired fatty acids (e.g., DHA and/or EPA), 5-80% of one or more desired fatty acids (e.g., DHA and/or EPA), 5-70% of one or more desired fatty acids (e.g., DHA and/or EPA), 5-60% of one or more desired fatty acids (e.g., DHA and/or EPA), 5-50% of one or more desired fatty acids (e.g., DHA and/or EPA), 5-40% of one or more desired fatty acids (e.g., DHA and/or EPA), 5-30%, of one or more desired fatty acids (e.g., DHA and/or EPA), or 5-20% of one or more desired fatty acids (e.g., DHA and/or EPA). It will be recognized that the lower limit of these ranges can be 10%, 20%, 30%, 40%, 50%, 60%, 70%, or 80% as appropriate. In some preferred embodiments, the phospholipid composition of the present invention comprise a mixture of different fatty acids in postion 1 as suggested above in combination with 18: 2 n-6 (LA) in position 2. LA can be present in position 2 in the range of 20-100%, 40-100%, 60-100% or 80-100%.

In preferred embodiments, the phospholipid compositions of the present invention are substantially free of organic solvents, comprise greater than about 10% DHA at position 1 (wherein position 1 can have a total of 100% of a mixture of fatty acid residues attached) and preferably from about 10% to about 50% DHA at position 1. As described, in preferred embodiments, the phospholipid products of the present invention are substantially free of organic solvents compared to other synthetic phospholipids. In more preferred embodiments, the phospholipid compositions of the present invention contain no organic solvents. Traces of organic solvents are hard to remove and they pose a significant health risk even in low concentration to humans, especially infants. Consequently, the synthetic marine phospholipids disclosed in this invention are safe to be orally administrated by a human.

Marine phospholipids can be extracted from natural sources such as marine species as well. Such natural marine phospholipids have EPA/DHA distributed mainly in position 2. In contrast, in preferred embodiments, the synthetic marine phospholipids of the present invention contain DHA, EPA, or other omega-3 fatty acids in position 1 and are substantially free of DHA and EPA at position 2. This is because the normally occurring fatty acids present at position 2 in the starting phospholipids prior to transesterification are retained. By “substantially free,” it is meant that position 2 contains less than 5% DHA and/or EPA, and preferably less than 1% DHA and/or EPA.

Marine phospholipids extracted from marine sources have a characteristic smell and taste of rancid fish. The GC profile of the volatiles confirms the presence of these degradation products, such as short chain aldehydes and carboxylic acids. In preferred embodiments, the synthetic marine phospholipid compositions of the present invention are substantially free of volatile organic compounds and are therefore much more suitable as a food supplement for humans and animals. Accordingly, in preferred compositions, the present invention provides synthetic marine phospholipids compositions having high or increased palatability, wherein the high or increased palatability is due to low levels of organic solvents and/or volatile organic compounds. In preferred embodiments, palatability is assayed by feeding the composition to a panel of subjects, preferably human. In more preferred embodiments, the phospholipids compositions have high or increased palatability as compared to naturally extracted marine phospholipids. In other preferred embodiments, the synthetic marine phospholipids compositions of the present invention are safe for oral administration.

In other preferred embodiments, synthetic marine phospholipids are used to fortify food products like pet food, cakes, chocolate and bread. In some more preferred embodiments, the phospholipids are utilized as emulsifiers in food products such as mayonnaise. The positive health effects of omega-3 fatty acids in the area of cardiovascular disease, cancer, inflammation and psychosomatic disorders are well documented, as well as positive effects on the brain and retina (M. A. Moyad; Urologic Oncology 23 (2005) 23-28; M. A. Moyad; Urologic Oncology 23 (2005) 36-48). Therefore, by adding marine phospholipids to the food, the nutritional value would increase without compromising the quality of the food compared to their natural analogues and fish oil. Synthetic marine phospholipids have less distinct smell and taste of fish than extracted marine phospholipids and are more stable than fish oil. The nutritional value would be even greater than food enriched with fish oil due to the increased bioavailability of EPA and DHA when attached to a glycerophospholipid backbone (D. Lemaitre-Delaunay, C. Pachiaudi, M. Laville, J. Pousin, M. Armstrong and M. Lagarde, J. Lipid. Res. 40 (1999) 1867; V. Wijendran, M. Huang, G. Diau, G. Boehm, P. W. Nathanielsz and J. T. Brenna; Pediatr. Res. 51 (2002) 256). Therefore, due to the increased stability, the improved organoleptic properties and bioavailability marine phospholipids can be used to fortify food, in addition used as a food supplement. In still other embodiments, the synthetic marine phospholipids are utilized as pharmaceuticals, elderly food and pregnancy food. Further more, marine phospholipids may form liposomes in aqueous solutions and can therefore be used as drug carriers for targeted drug release. In yet another preferred embodiments, synthetic marine phospholipids are added to animal feed in order to improve the nutritional value of the agricultural products derived from the animal. For example, laying hens could be fed marine phospholipids in order to produce egg fortified with omega 3-fatty acids.

In some preferred embodiments, synthetic marine phospholipids can replace extracted phospholipids in the area of aquaculture, e.g. for feeding fish at different stages. For example it can be used to enrich prey organism such as artemia and rotifer with DHA. Prey organisms with elevated levels of DHA are a beneficial feed for larvae of fish including, but not limited to cod, halibut, gilthead seabream, crustacean and mollusk in order to promote growth and reduce malformations (U.S. Pat. No. 6,789,502). In addition, synthetic marine phospholipids can be included in the fish feed for fish larvae, adult and juvenile fish. Thereby, reducing malformation, improving fecundity, improving hatchability of fish eggs and improving growth and overall survival rate. This invention discloses that the marine phospholipid composition can be used successfully to enrich prey organisms in such a way that the fish larvae feeding on them grow quicker. In addition, have reduced malformations and contain more EPA/DHA.

In still other embodiments, enzymatically synthesized marine phospholipids can be used to improve the bioavailability of nutritionally important fatty acids such as EPA and DHA. This invention discloses that a higher levels of DHA in the brain of growing rat pups can be obtained by feeding with the composition described above (DHA attached to position 1 in the PL molecule) compared to fish oil and natural extracted marine phospholipids containing DHA in position 2 (p<0.1). High levels of DHA have been associated with improved cognitive performance. This invention also discloses that the DHA attached to position 1 in a PL molecule was more efficient in reducing arachidonic acid levels in plasma compared to fish oil (p<0.05). In both experiments the rats were given the same amount of DHA, the difference was in the form the DHA was given to the animals i.e. phospholipids versus triglycerides. AA can be a precursor in the formation of pro-inflammatory prostaglandins; therefore the reduction of AA is a common target for reducing inflammation in a number of conditions such as cardiovascular disease, rheumatoid arthritis, cancer and Alzheimer's disease.

### EXAMPLES

### Example 1

The commercial product Alcolec 40P® from American Lecithin Company Inc (Oxford, Conn., USA) was used as a phospholipids starting material. This is a crude soybean phospholipid product containing 40% PC, 26% phosphatidylethanolamine, 11% phosphatidylinositol, 1% phosphatidylserine, 13% phytoglycolipids as well as 14% other phosphatides (w/w). A fatty acid ethyl ester (FAEE 10-50) which contained 10% EPA and 50% DHA (relative GC peak areas) was used as an acyl donor. All reactions were performed under N₂ at atmospheric pressure and at 55° C. The reaction time was varied from 1 to 140 hours. In order to analyze the product, the sample was fractionated by HPLC-UV with a silica column and methanol-water as mobile phase. The isolated PC fraction was then dried under nitrogen prior to derivatization, finally the fatty acid profile was determined by analyzing the derivatives on a gas chromatography-flame ionization detector (GC-FID). Furthermore, the relationship between PC, LPC and GPC was determined using HPLC with the method above, except that the UV detector was replaced by an evaporative light scattering detection (ELSD). Integrated ELSD peak areas were reported for PC/LPC/GPC (total 100%) and other PL species were not analyzed.

In order to obtain the final product the enzymes were removed by filtration. Then, residual amines were removed by increasing the temperature and reducing the pressure. Finally, a triglyceride carrier was added to the product, followed by the removal of the residual free fatty acids and/or esters by short path distillation.

10 g of Alcolec 40P was mixed with 30 g of FAEE 10-50, 10 g of TL-IM and 0.3 g of ethanolamine. The reaction was terminated after 24 hours. The results showed that the PC fraction contained 5.5% EPA+DHA. As a reference, Alcolec 40P, TL-IM and FAEE 10-50 were mixed under identical conditions. The isolated PC fraction from this sample contained 2.6% EPA+DHA.

### Example 2

10 g of Alcolec 40P was mixed with 30 g of FAEE 10-50, 10 g of TL-IM and 0.3 g of triethylamine. The reaction was terminated after 48 hours. The results showed that the PC fraction contained 6% EPA and 17% DHA. As a reference, Alcolec 40P, TL-IM and FAEE 10-50 was mixed under identical conditions. The isolated PC fraction from this sample showed 2.8% EPA and 2.8% DHA.

### Example 3

The experiment was performed under identical conditions as in example 1 except that for the amount of enzyme was reduced to 5 g for both samples. The reaction was terminated after 4 days. The isolated PC fraction showed 2.5% and 0.9% EPA+DHA for the reaction with ethanolamine addition and the reference sample, respectively.

### Example 4

The experiment was performed under identical conditions as in example 1. After the reaction was terminated the enzymes was filtered off and reused in a new batch under identical conditions. The rate of transesterification of the second batch was 66% of the first batch. The same experiment was performed without ethanolamine addition; the rate of transesterification in the second batch was now only 30% of the first batch.

### Example 5

The experiments were performed by mixing 30 g FAEE 10-50, 10 g Alcolec 40P, 7.5 g TL-IM (1% water content) with a number of different basic compounds (each 5 mmol) (Table 1) in a glass flask using either a magnetic stirrer (45° C.) (row 1-10) or in a shaker incubator (45° C.) (row 11-16). The level of EPA/DHA esterified to the combined fraction of PC+LPC after 24, 48 and 72 hours can be seen in Table 1.

TABLE 1
Effect of amine addition of EPA/DHA incorporation.

| | 24 h | | 48 h | | 72 h | |
|---|---|---|---|---|---|---|
| Substance | % EPA | % DHA | % EPA | % DHA | % EPA | % DHA |
| No addition | 0.4 | 0.2 | 0.5 | 0.3 | 0.8 | 0.5 |
| Aniline | 0.3 | 0.1 | 0.5 | 0.4 | 1.5 | 1.1 |
| Methyl aniline | 0.3 | 0.2 | 0.4 | 0.3 | 0.9 | 0.6 |
| Dimethyl | 0.2 | 0.1 | 0.4 | 0.2 | 0.6 | 0.4 |
| aniline | | | | | | |
| Benzylamine | 0.4 | 0.3 | 1.1 | 0.7 | 1.6 | 1.3 |
| Isobutylamine | 0.8 | 0.6 | 1.3 | 1.1 | 1.9 | 1.6 |
| Butylamine | 0.8 | 0.5 | 1.4 | 1.1 | 1.6 | 1.4 |
| Tripropylamine | 0.7 | 0.5 | 1.0 | 0.9 | 2.1 | 1.4 |
| Triethylamine | 1.7 | 1.5 | 2.6 | 2.8 | 4.1 | 4.1 |
| Diethylamine | 1.7 | 1.6 | 3.2 | 3.4 | 6.2 | 6.3 |
| NaOEt | 1.1 | 1.0 | 2.5 | 2.7 | 4.1 | 4.8 |
| KOEt | 0.5 | 0.4 | 1.4 | 1.4 | 2.8 | 3.1 |
| Caffeine | 0.3 | 0.2 | 0.9 | 0.6 | 1.2 | 0.8 |
| Nicotine | 0.5 | 0.4 | 1.1 | 0.9 | 1.7 | 1.6 |
| Choline | 0.6 | 0.2 | 0.8 | 0.4 | 1.0 | 0.7 |
| Chloride | | | | | | |
| Ethanolamine | 1.1 | 0.8 | 2.1 | 1.9 | 4.0 | 5.0 |

### Example 6

The experiment was performed as in example 5 except that only triethylamine and ethanolamine were tested. The amount amine added to the reaction mixture varied from 3-11% (w/w) relative to the amount phospholipid. The reaction was terminated after 72 hours and the results are shown in Table 2 below.

TABLE 2
Transesterification efficiency as a function of amount amine added

| Substance | Amount | PC/LPC/GPC | EPA (72 h) | DHA (72 h) |
|---|---|---|---|---|
| Triethylamine | 3% | 45/39/16 | 3.3 | 3.3 |
| Triethylamine | 5% | 41/42/17 | 3.8 | 4.0 |
| Triethylamine | 7% | 34/41/28 | 3.9 | 4.1 |
| Triethylamine | 9% | 34/43/23 | 4.2 | 4.7 |
| Triethylamine | 11% | 31/46/23 | 4.5 | 5.0 |
| Ethanolamine | 3% | 37/39/24 | 1.8 | 2.0 |
| Ethanolamine | 5% | 29/37/44 | 1.4 | 1.8 |
| Ethanolamine | 7% | 23/35/42 | 0.8 | 1.0 |
| Ethanolamine | 9% | 27/35/38 | 0.5 | 0.7 |
| Ethanolamine | 11% | 42/40/18 | 0.6 | 0.5 |

### Example 7

Transesterification according to the method outlined in [5] was performed using either 99% PC, 40% PC or 40% PC+triethylamine (TEA) as starting materials. The purpose was to investigate the effect of purity on reaction rate and the ability of amine addition to compensate for the lowering of reaction rate by the more impure starting material. 1 g PC from egg (99%) were mixed with 300 mg of RM-IM and 3 g of 50-21 EPA/DHA as free fatty acids using a shaker incubator at 65° C. Furthermore, the experiment was repeated under the same conditions using 40% PC from soy bean instead of 99% PC. Finally the experiment was repeated using 40% PC and 3% (w,w) addition of triethylamine. In all 3 experiments the reaction time was 72 hours. The amount EPA/DHA attached to the PC+LPC fraction and the relationship between PC/LPC/GPC (% ELDS peak area) can be seen in Table 3.

TABLE 3
The effect of purity of starting material on rate of transesterification

| | | | Soy lecithin |
|---|---|---|---|
| Substrate | 99% egg PC | Soy lecithin (40% PC) | (40%) PC + TEA |
| PC/LPC/GPC | 71/27/2 | 63/16/21 | 50/19/31 |
| EPA/DHA | 11.8/2.0 | 7.4/1.1 | 11.1/1.7 |

### Example 8

Four different phospholipid compositions (MPL1-MPL4) were tested as emulsifiers in bread and added to the dough during baking. The tested phospholipids had the following compositions (Table 4):

TABLE 4
Compositions used as emulsifiers in loaf bread.

| | | | EPA/DHA |
|---|---|---|---|
| Treatment | PC/LPC/GPC* | EPA/DHA (total)** | (PC + LPC)** |
| MPL 1 | 42/40/18 | 12.5/6.9 | 7.6/7.3 |
| MPL 2 | 11/39/50 | 6/15 | 10.3/13.7 |
| MPL 3 | 100/0/0 | 12.5/8.7 | 35.0/12.0 |
| MPL 4 | 60/30/5 | 4.2/8.7 | 3/2 |

*Relationship between PC/LPC/GPC, for simplicity other phosphatides are not analyzed.
**The column EPA/DHA (total) shows the EPA/DHA level in both PL and TG combined, whereas the column EPA/DHA (PC + LPC) shows EPA/DHA level on PC + LPC isolated by preparative HPLC.

Treatments MPL 1 and MPL 2 were prepared using any of the previous examples except that no base was added to the reaction mixtures. MPL 3 (Krill oil extract) was obtained from Neptune Biotech (Laval, Quebec, Canada). Treatment MPL 4 was prepared using the method described [5], in this method no base was added and 96% pure soy PC was used as starting material. MPL 1, MPL 2 and MPL 4 contained 30% triglycerides, whereas MPL 3 contained 50% triglycerides. The prepared bread products (loaf) were tested for palatability by a panel of 9 human subjects. The human subjects were then questioned about the palatability of each of the four compositions, and in particular about the odor, flavor, texture and visual impression of the final product. It was found that MPL 3, the extracted marine phospholipids, had a distinct fishy odor and flavor compared to the other treatments. There was no difference in odor and flavor between the other treatments. Headspace GC was used to analyze the presence of volatile organic compounds (VOCs) in the samples. It was found that MPL 3 had a significant higher amount these compounds compared to the other compositions and the VOCs present were characteristic of those resulting in the smell/taste of rancid fish (short chain fatty acids and aldehydes). There were found no differences in texture between the 4 treatments. However it was found a difference in visual impression. The bread baked with MPL 3 was colored pink, and the bread baked with MPL 1, 2 and 4 was colored slightly grey. It was also found using headspace GC that MPL 4 contained a significant amount of chloroform, due to the use of cholorform in the preparation of this product. However, in the final bread product supplemented with MPL 4, no traces of chloroform could be found.

### Example 9

Five different lipid compositions (Table 5) were prepared and used as enrichment medium for the cultivation of rotifers (Brachionus plicatilis) and artemia (Artemia salina). The prey organisms were fed to a culture of gilhead sebream during a period of 55 days. The growth rate of the fish larvae was recorded and finally the level of malformation in the fish was observed visually and by the use of X-ray.

TABLE 5
Composition of the enrichment diets.

| Treatment | PC/LPC/GPC* | EPA/DHA (total)** | EPA/DHA (PCs)** |
|---|---|---|---|
| NAT501 | 53/37/10 | 7/22 | 4/3 |
| NAT502 | 20/37/43 | 6/15 | 10/14 |
| NAT503 | 20/37/43 | 10/28 | 10/14 |
| NAT504 | 100/0/0 | 13/12 | 12/26 |
| NAT505 | 26/42/12 | 5/18 | 0/0 |

*Relationship between PC/LPC/GPC, for simplicity other phosphatides are not analyzed.
**The column EPA/DHA (total) shows the EPA/DHA level in both PL and TG combined, wheras the column EPA/DHA (PC) shows EPA/DHA level on PC + LPC isolated by preparative HPLC.

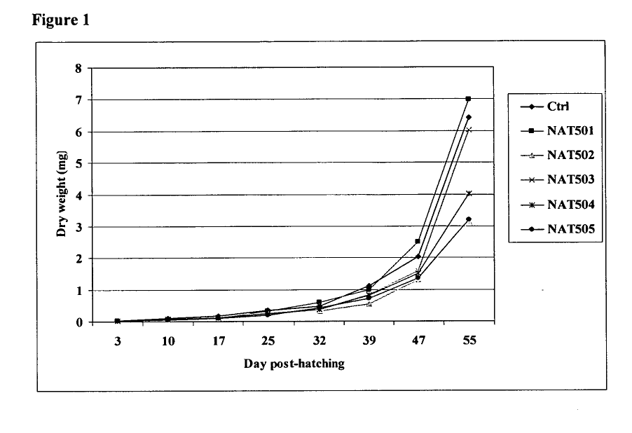

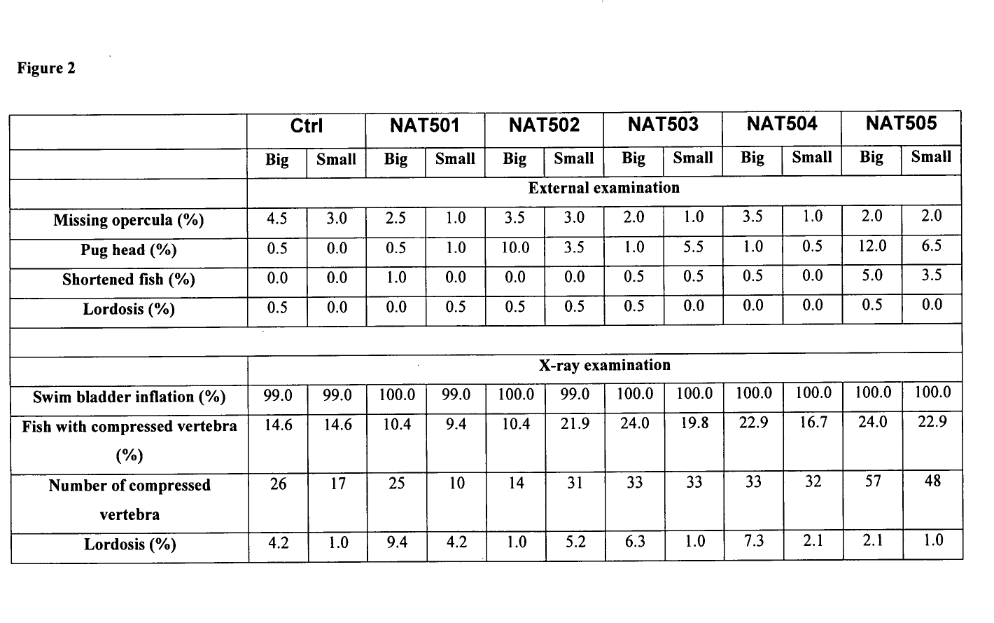

Treatment NAT501, NAT502, NAT503 and NAT505 were prepared according to any of the methods described above except that no bases were added to the reaction mixtures. The treatments consisted of 30% triglycerides carrier, except for NAT 504 which consisted of 70% triglycerides. NAT504 consisted of naturally occurring marine phospholipids and was prepared by extracting the PLs from fish meal using ethanol. As a control, DHA Protein Selco (for the rotifers) and Easy Selco (for the artemia) (products of INVE, Belgium) were used for the enrichment of rotifers and artemia, respectively. The prey organism were enriched with the treatments for a 24 hours then stored at 6° C. until use. 3 days after hatching the gilthead seabream fish larvae were fed rotifers and after 26 days the diet was switched to artemia until day 47 when the experiment was terminated. The fish larvae were weighed at certain time intervals during the experiment (FIG. 1). In the end samples of the fish larvae were freeze dried and the fat was extracted using supercritical fluid extraction (SFE) under the following conditions: pure CO₂, 45 minutes extraction time, 100° C. extraction temperature, 100° C. restrictor temperature and 7500 psi extraction pressure. The level of EPA/DHA in the extract was measured using GC-FID and the results are shown in Table 6. The malformations were also determined, by visual inspection and X-ray analysis (FIG. 2).

After 55 days the fish larvae on an average weighed 6.41 mg, 6.98 mg, 3.13 mg, 6.05 mg, 4.02 mg and 3.20 mg after feeding on prey organisms enriched with control, NAT501, NAT502, NAT503, NAT504 and NAT505, respectively.

TABLE 6
Content EPA/DHA in the triglyceride in gilthead seabream at day 55.

| Fatty | Con- | | | | | |
|---|---|---|---|---|---|---|
| acid | trol | NAT501 | NAT502 | NAT503 | NAT504 | NAT505 |
| DHA | 11.3 | 22.3 | 10.8 | 19.7 | 8.3 | 7.6 |
| (%) | | | | | | |
| EPA | 5.5 | 8.2 | 7.2 | 12.4 | 4.3 | 7.2 |
| (%) | | | | | | |

Furthermore, the number of surviving fish in the different groups were 24189 (control), 12230 (NAT501), 9700 (NAT502), 5752 (NAT503), 9504 (NAT504) and 8544 (NAT505). The commercially available control diet contained all necessary nutrients, whereas NAT501-NAT503 and NAT505 did not contain vitamin A and vitamin D.

### Example 10

The bioavailability of different forms of DHA was investigated by measuring the transfer of DHA into the brain of newly weaned Sprague-Dawley rats after 10 days of feeding. The treatments tested were control, eMPL, nMPL1, nMPL2 and DHA-TG (see Table 8A for fatty acid composition). All the treatments were balanced for DHA (n-3), 18:2 (n-6), 18:3 (n-3) and for the total amount of fatty acid. The control was obtained by mixing linseed oil and ethyl esters of soy bean oil and DHA-TG (tuna oil) was obtained from Berg Lipid Tech (Ålesund, Norway), both contained 0% phospholipids. EMPL was obtained by extracting marine phospholipids from fish meal using ethanol, nMPL1 and nMPL2 were prepared using the methods described in the previous examples, except that no base was added. Treatments nMPL1, nMPL2 and eMPL all contained 30% PL, however the degree of hydrolysis was different (see Table 8B for details). Finally, the balanced treatments were mixed with skimmed milk (1:10) and consumed by the rats for 10 days (day 20-30 post weaning). The milk samples (10% fat) containing DHA were fed ad libitum to the rats (N=5) during the last phase of the brain growth spurt (day 20-30). The rats consumed 5 mL during the first two days, 7 mL during the following next two days, 9 mL during the following two days and 10 mL during the final 3 days. The pellet diet of pups contained no essential fatty acids (all fat was hydrogenated coconut oil). At an age of 30 days, the rats were sacrificed and their brain tissue dissected and frozen for gas chromatographic determination of DHA. The animals fasted the night before sampling. Three pups were sacrificed in the beginning of the test period to determine the base level of DHA in the brain.

TABLE 8A
Major fatty acids present in the materials used in the
bioavailability study (mg/g of total fatty acids).

| Fatty acid | Control | eMPL | nMPL1 | nMPL2 | DHA-TG |
|---|---|---|---|---|---|
| 18:2 (n-6) | 200.0 mg/g | 199.6 mg/g | 200.6 mg/g | 199.8 mg/g | 200.0 mg/g |
| 18:3 (n-3) | 40.0 mg/g | 39.9 mg/g | 39.9 mg/g | 40.2 mg/g | 40.0 mg/g |
| 22:6 (n-3) | 0 mg/g | 60.1 mg/g | 60.1 mg/g | 60.0 mg/g | 59.9 mg/g |

TABLE 8B
Phospholipid composition of the materials

| | eMPL | nMPL1 | nMPL2 |
|---|---|---|---|
| PC/LPC/GPC* | 100/0/0 | 43/34/23 | 53/37/10 |

*Relationship between PC species determined by HPLC-ELSD

The levels of DHA in the brain of the rat pups were found (N=5) to be the following 13.78%, 15.09%, 15.41%, 15.34%, 15.08% and 13.35% for control, eMPL, nMPL1, nMPL2, DHA-TG and baseline, respectively. The results obtained show that DHA attached to a phospholipid in position sn-1 is incorporated more efficiently to the brain of rat pups than DHA bound to either triglycerides or phospholipids in position sn-2 (p<0.1).

### Example 11

The omega-3 fatty acids EPA and DHA can competitively inhibit n-6 arachidonic acid (n-(AA) metabolism and thus reduce the generation of inflammatory 4-sereis leukotrienes and 2-series prostaglandin mediators (T. H. Lee, R. L. Hoover, J. D. Williams, R. I. Sperling, J. Ravlese III, B W Spuir, D. R. Robinson, E. J. Corey, R. A. Lewis and K. F. Austen. N Engl J Med; 312 (1985) 217). Omega-3 fatty acids have therefore been promising in the treatment of inflammatory disorders such as osetoarthritis, rheumatoid arthritis and atherosclerosis. In example 10, at day 30 blood samples were drawn in a heparinized 5 ml syringe (23 G needle), and plasma and red blood cells were separated before analyzed by GC-FID. The results showed that for the rats feeding on control, eMPL, nMPL1, nMPL2 and DHA-TG the total AA levels in plasma were 22.9%, 9.9%, 15.3%, 14.9% and 16.3%, respectively. The total AA level in red blood cells (RBC) after feeding on control, eMPL, nMPL1, nMPL2 and DHA-TG were 21.9%, 16.1%, 17.7%, 17.9% and 18.0%, respectively. The results clearly show that both extracted marine phospholipids and their enzymatically synthesized analogues (nMPL1 and nMPL2) are efficient for reducing the total AA levels in plasma and RBC compared to control (p<0.01) and fish oil (p<0.1).

What should be clear from above is that the present invention provides novel methods for modifying phospholipids and novel compositions resulting from the described methods. All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in medicine, biochemistry, or related fields are intended to be within the scope of the following claims.

1. A process for modifying phospholipid material which comprises exchanging acyl groups in a phospholipid by enzymatic exchange with a free fatty acid or ester, the reaction mixture comprising an immobilized lipase and a cationic compound, wherein the cationic compound enhances the enzymatic activity of the immobilized lipase.

2. A process as claimed in claim 1, wherein the cationic compound is an organic molecule with an amine functional group.

3. A process as claimed in claim 2, wherein the cationic compound is present in the range of 0.1-10% relative to the phospholipid (w/w).

4. A process as claimed in claim 3, wherein the organic molecule containing an amine functional group is triethylamine or ethanolamine.

5. A process as claimed in claim 4, wherein the acyl donor is a fatty acid ethyl ester containing EPA or DHA.

6. A process as claimed in claim 5, wherein the phospholipid starting material is a naturally occurring soybean lecithin.

7. The process of claim 1, wherein the reaction is substantially solvent-free.

8. A composition produced by the method of claim 1, wherein said composition comprises phospholipids having a DHA or EPA residue at position 1 of the phospholipid.

9. The process of claim 1, further comprising the step of supplementing a food product with the modified phospholipid.

10. The process of claim 1, further comprising the step of formulating a pharmaceutical composition with the modified phospholipid.

11. The process of claim 1, further comprising the step of supplementing an animal feed with the modified phospholipid.

12. The process of claim 1, further comprising the step of supplementing an infant formula with the modified phospholipid.

13. The process of claim 1, further comprising the step of formulating the modified phospholipids for oral administration.

14. An oral delivery vehicle comprising the composition of claim 8.

15. A food product comprising the composition of claim 8.

16. A pharmaceutical composition comprising the composition of claim 8.

17. An animal feed comprising the composition of claim 8.

18. A composition comprising phospholipids having the following structure: 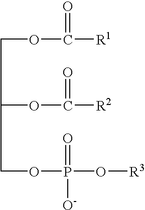
wherein R1 is a fatty acid, R2 is OH or a fatty acid, and R3 is H or choline, ethanolamine, inositol or serine, said composition having at least 10% of a combination of DHA and EPA at position R1 and being substantially free of EPA and DHA at position R2.

19. The composition of claim 18, wherein said composition contains from about 10% DHA to about 50% DHA at position R1.

20. The composition of claim 18, wherein said composition contains from about 10% DHA to about 40% DHA at position R1.

21. The composition of claim 18, wherein said composition contains from about 10% DHA to about 30% DHA at position R1.

22. The composition of claim 18, wherein said composition contains from about 10% DHA to about 20% DHA at position R1.

23. The composition of claim 18, wherein said composition contains from about 10% EPA to about 50% EPA at position R1.

24. The composition of claim 18, wherein said composition contains from about 10% EPA to about 40% EPA at position R1.

25. The composition of claim 18, wherein said composition contains from about 10% EPA to about 30% EPA at position R1.

26. The composition of claim 18, wherein said composition contains from about 10% EPA to about 20% EPA at position R1.

27. The composition of claim 18, wherein said composition is at least about 50% acylated at positions R1 and R2.

28. The composition of claim 18, wherein said composition contains from about 5% to about 75% of a linoleic acid isomer residue at position R2.

29. The composition of claim 18, wherein said composition contains from about 5% to about 50% of a linoleic acid isomer residue at position R2.

30. The composition of claim 24, wherein said linoleic acid isomer residue is selected from the group consisting of 9,12-ocadecadienoic acid, 9,11-ocadecadienoic acid, 10,12-ocadecadienoic acid, 8,10-octadecadienoic acid, and 11,13-octadecodienoic acid and combinations thereof.

31. The composition of claim 18, wherein said composition comprises less than about 5% EPA or DHA a position R2.

32. The composition of claim 18, wherein said composition comprises less than about 1% EPA or DHA a position R2.

33. The composition of claim 18, wherein said composition is substantially free of organic solvents.

34. A food product that is safe to be taken orally by humans in a concentrated form comprising the composition of claim 18.

35. An animal feed comprising the composition of claim 18.

36. A pharmaceutical composition comprising the composition of claim 18.

37. A composition comprising synthetic phospholipids having the following structure: 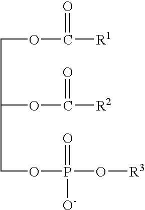
wherein R1 is a fatty acid, R2 is OH or a fatty acid, and R3 is H or choline, ethanolamine, inositol or serine, said composition characterized in having high palatability in terms of at least one of smell, taste, aftertaste, and mouthfeel or combinations thereof.

38. The composition of claim 37, wherein said high palatability is in comparison to at least one of naturally extracted marine phospholipids and synthetic phospholipids prepared with organic solvents.

39. The composition of claim 37, wherein said palatability is determined by a panel of human subjects.

40. A safe and palatable synthetic marine phospholipid composition characterized in being substantially free of at least one of organic solvents and volatile organic compounds.

41. A composition providing increased bioavailability of long chain fatty acids comprising phospholipids having the following structure: 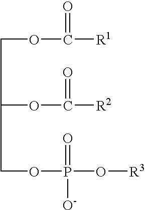
wherein R1 is a fatty acid, R2 is OH or a fatty acid, and R3 is H or choline, ethanolamine, inositol or serine, said composition enriched for DHA or EPA at position R1 as compared to position R2.

42. The composition of claim 41, wherein said composition has at least 10% DHA at position R1 and being substantially free of EPA and DHA at position R2.

43. A method of increasing the bioavailability of EPA or DHA comprising:
providing phospholipids having the following structure: 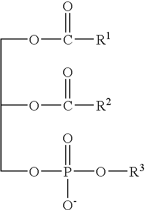
wherein R1 is a fatty acid, R2 is OH or a fatty acid, and R3 is H or choline, ethanolamine, inositol or serine, said composition enriched for DHA or EPA at position R1 as compared to position R2 and administering said composition to a subject under conditions such that bioavailabilty of EPA or DHA to said subject is increased as compared to compositions enriched for EPA or DHA at position R2.

44. A method of treating inflammation in a subject comprising:
a) providing a phospholipid composition comprising DHA, EPA or a combination thereof, and
b) administering said phospholipids composition to a subject under conditions such that inflammation is said subject is reduced.

45. The method of claim 44, wherein said phospholipid composition is the composition of claim 18.

46. The method of claim 44, wherein said phospholipid composition is extracted from natural sources.

47. The method of claim 44, wherein said subject is a human.

48. The method of claim 44, wherein said subject is an animal.

49. A method of producing prey organisms for use in aquaculture, said method comprising cultivating said organisms during at least part of their life cycle in an aqueous medium comprising the composition in claim 18.
